(19) United States
(12) Patent Application Publication
(10) Pub. No.: US 2024/0189696 A1
(43) Pub. Date: Jun. 13, 2024

(54) SCREEN GOLF DEVICE

(71) Applicants: GSPI CO., LTD., Daejeon (KR); SBS MEDIANET CO., LTD., Seoul (KR)

(72) Inventors: San Bok LEE, Daejeon (KR); Hong Rak CHOI, Seoul (KR)

(73) Assignees: GSPI CO., LTD., Daejeon (KR); SBS MEDIANET CO., LTD., Seoul (KR)

(21) Appl. No.: 18/550,641

(22) PCT Filed: Mar. 14, 2022
(86) PCT No.: PCT/KR2022/003553

§ 371 Date: Sep. 14, 2023

(30) Foreign Application Priority Data
Mar. 16, 2021 (KR) 10-2021-0034319
Apr. 21, 2021 (KR) 10-2021-0051851

(51) Int. Cl. A63B 71/06; A63B 24/00; A63B 102/32

(52) U.S. Cl. CPC A63B 71/0622; A63B 24/0062; A63B 2024/0096; A63B 2071/0636; A63B 2102/32

(57) ABSTRACT

The present invention relates to a screen golf device including a virtual game storage unit and a processing unit, wherein the virtual game storage unit stores data for virtual player characters of actual professional golfers and condition data of past golf games played by at least one of the actual professional golfers. When a user strikes a golf ball on a tee box with a golf club on a swing plate, the processing unit derives the trajectory of the golf ball based on golf ball sensing information obtained from a sensing unit sensing the movement of the golf ball, processes the virtual golf game between the virtual player character and the user based on the derived trajectory and the condition data of the past golf game, and displays the progress of the virtual golf game on a screen unit.

### TECHNICAL FIELD

The present invention relates to a screen golf device.

### BACKGROUND ART

In general, golf is a sport in which a golfer hits a white ball on a course with a club into a hole, and superiority is determined based on the number of strokes taken from the starting point to putting the ball in the hole.

That is, a hole cup is set at a certain distance away, and a golfer strikes the ball with a club such as a driver to put the ball in the hole cup. Each course has a set number of strokes, and the golfer who completes the course with the fewest strokes wins.

As golf has become popularized, not only in the field but also indoors, screen golf courses are prevalent.

Additionally, there is an increasing demand for enhancing the sense of reality at screen golf courses.

### DISCLOSURE

### Technical Problem

A screen golf device according to an embodiment of the present invention is to provide a screen golf game that offers a realistic experience to users.

The objects of the present invention are not limited to those mentioned above, and other objects not mentioned herein will be clearly understood by those skilled in the art from the following description.

### Technical Solution

To accomplish the above-mentioned objects, according to the present invention, there is provided a screen golf device including a virtual game storage unit and a processing unit, wherein the virtual game storage unit stores data for virtual player characters of actual professional golfers and condition data of past golf games played by at least one of the actual professional golfers. When a user strikes a golf ball on a tee box with a golf club on a swing plate, the processing unit derives the trajectory of the golf ball based on golf ball sensing information obtained from a sensing unit sensing the movement of the golf ball, processes the virtual golf game between the virtual player character and the user based on the derived trajectory and the condition data of the past golf game, and displays the progress of the virtual golf game on a screen unit.

The virtual game storage unit stores relay voice data, the relay voice data includes voice data of broadcasters for predetermined events of the virtual golf game, the voice data of the broadcasters is generated by synthesizing voice information of broadcasters who relayed the past golf game, and the processing unit processes the voice data of the broadcasters when the events occur during the processing of the virtual golf game.

The condition data of the past golf game includes wind direction data and wind speed data of the past golf game, and the processing unit changes the derived trajectory based on at least one of the wind direction data and the wind speed data.

The processing unit processes the virtual golf game in the virtual course implementing a teeing ground, a through the green, a green, and hazards that constitute the holes of the past golf game.

The processing unit applies a handicap to the virtual player character in the virtual golf game, to enable the user to compete with the virtual player character of the real professional golfer.

The processing unit sets revenue for the actual professional golfer in proportion to the frequency of selection of the virtual player character by the user as an opponent in the virtual golf game.

The processing unit registers the user's identification information and the number of strokes for each round in the virtual Hall of Fame when the user defeats the virtual player character of the actual professional golfer in the virtual golf game.

The processing unit reads advertisement data featuring actual professional golfers who participate in the virtual golf game from the virtual game storage unit and displays the advertisement data on the screen unit.

The screen golf device according to an embodiment of the present invention can provide a game on a virtual golf course similar to a golf course where a professional golfer played actually, together with a virtual player character of the real professional golfer, thereby allowing the user to enjoy a realistic screen golf experience.

The effects of the present disclosure are not limited to the above-mentioned effects, and other effects, which are not specifically mentioned herein, will be clearly understood by those skilled in the art from the following description.

### DESCRIPTION OF DRAWINGS

FIG. 1 illustrates a screen golf device according to an embodiment of the present invention.

FIG. 2 is for depicting an example of a method for deriving the trajectory of a golf ball.

FIG. 3 is for depicting a change in trajectory of the golf ball depending on wind direction and wind speed of the condition data of a past golf game.

### MODE FOR INVENTION

Hereinafter, embodiments of the present invention will be described in detail with reference to the accompanying drawings. However, the accompanying drawings are only described in order to more easily disclose the contents of the present invention, and it will be easily understood by those of ordinary skill in the art that the scope of the present invention is not limited to the scope of the accompanying drawings.

The terms used in the following description are intended to merely describe specific embodiments, but not intended to limit the invention. An expression of the singular number includes an expression of the plural number, so long as it is clearly read differently.

The terms such as “include” and “have” are intended to indicate that features, numbers, steps, operations, elements, components, or combinations thereof used in the following description exist and it should thus be understood that the possibility of existence or addition of one or more other different features, numbers, steps, operations, elements, components, or combinations thereof is not excluded.

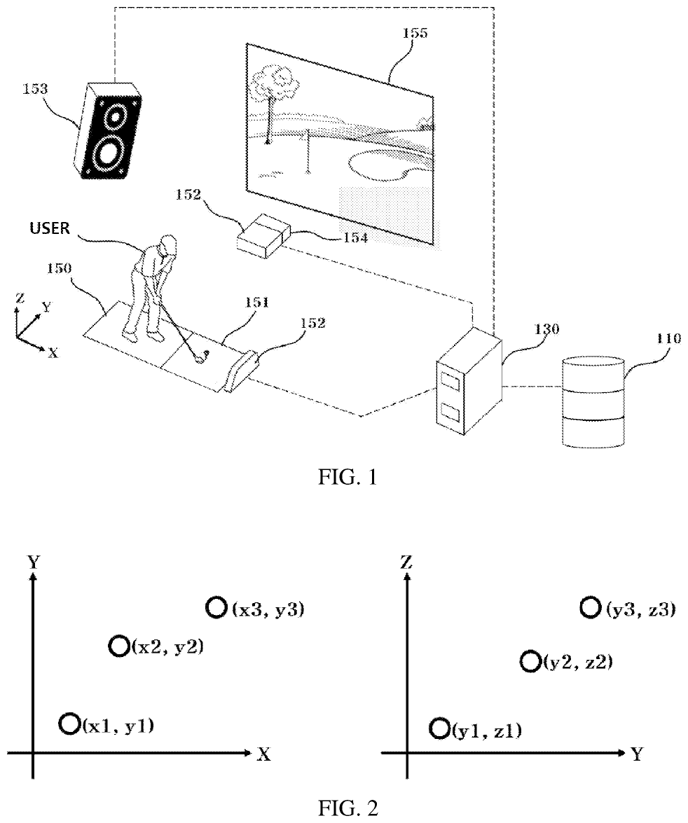

FIG. 1 illustrates a screen golf device according to an embodiment of the present invention. As illustrated in FIG. 1, the screen golf device according to an embodiment of the present invention includes a virtual game storage unit 110 and a processing unit 130.

The virtual game storage unit 110 stores data related to a virtual player character of a real professional golfer and condition data of a past golf game that at least one among real professional golfers played.

The real professional golfer may include at least one of a retired professional golfer and a current professional golfer. For example, the real professional golfer can include professional golfers who have participated or are currently participating in domestic and international professional golf games, such as Tiger Woods, Phil Mickelson, Park Seri, and Park Inbee.

Past golf games may include golf games in which the real professional golfer actually participated as a player. For example, Tiger Woods' past golf games could be golf games such as the PGA Tour British Open or US Open, which Tiger Woods participated in and won in 2000, and the Masters in 2001 and the US Open in 2002.

At this time, past golf games may include at least some of the games in which the real professional golfer actually participated.

The condition data of the past golf games may include golf course information where past golf games were played, course information of past golf games, topographical information about the course of the past golf games, weather information at the time of the past golf games, and round shot tracker information of the real professional golfer, but is not limited thereto.

The golf course information where the past golf game was played may include the name of the golf course, the location of the golf course, and the landscape graphic information of the golf course, but is not limited thereto.

Course information of the past golf games may include a course map, location and shape of the teeing ground, location and shape of the through the green, location and shape of the green, location and shape of hazards, and landscape graphic information of the course.

The teeing ground is a starting area of each hole, and the through the green may be an area that combines fairway and rough. Moreover, the green may be an area where the grass is mowed short for putting, and the hazards may include bunkers and water hazards as obstacles installed within the course for difficulty or landscaping of the course.

Topographic information of the course may include elevation and slope, but is not limited thereto.

Weather information may include temperature, wind speed, wind direction, and humidity at the time of the past golf game, but is not limited thereto.

The virtual game storage unit 110 can provide a database necessary for the operation of the screen golf device of the present invention. For example, the database of the virtual game storage unit 110 may be built in association with real professional golfers, past golf games, and condition data, and may be built in association with other items not mentioned above.

In FIG. 1, it is illustrated that the processing unit 130 and the virtual game storage unit 110 are located in the same space, but the present invention is not limited thereto. For example, the processing unit 130 may be connected to the virtual game storage unit 110 through a wired or wireless network. Alternatively, the processing unit 130 and the virtual game storage unit 110 can be installed in a single housing to communicate through a data bus.

Meanwhile, the virtual player character may be generated by 3D rendering the face and body of a real professional golfer and representing the actual player's shots through motion capture. Information and data about the virtual player character can be stored in the virtual game storage unit 110. At this time, the real professional golfer and the actual player may be the same person or different people.

When a user located on the swing plate 150 hits a golf ball on the tee box 151 with a golf club, the processing unit 130 derives the trajectory of the golf ball according to the golf ball sensing information derived from a sensing unit 152 which senses the movement of the golf ball.

The processing unit 130 may include a processor (for example, a CPU, AP, GPU, microcontroller, etc.) and a memory unit (for example, an SSD, an HDD, a ROM, a RAM, etc.).

The processing unit 130 may include an interface (for example, an USB, an HDMI, a D-SUB, etc.) for connecting to at least one of the sensing unit 152, the speaker 153, the tee box 151, and the beam projector 154 illustrated in FIG. 1. The processing unit 130 may include a communication module (for example, a Bluetooth module, a mobile communication module, a Wi-Fi module, a wired LAN module, etc.) for communicating with at least one of the sensing unit 152, the speaker 153, the tee box 151, the beam projector 154, and other electronic devices.

The processing unit 130 may include an input unit (for example, a keyboard, a mouse, a stylus pen, a touch screen, a touch panel, etc.) for inputting information, and a display unit (for example, a CRT, an LCD, an OLED, etc.) for displaying the operation of the processing unit 130.

The processing unit 130 can operate as a server computing device, and in this case, can communicate with a client computing device (not shown) located in the screen golf field via a network. The client computing device can be connected to the sensing unit 152, the speaker 153, the beam projector 154, and the tee box 151, and can deliver operation requests to the processing unit 130.

The sensing unit 152 is to derive the trajectory of the golf ball. For this, the sensing unit 152 may include a camera. One of the sensing units 152 can be installed on the ceiling of the screen golf course to capture the X-Y plane in FIG. 1. Another one of the sensing units 152 can be installed on the floor of the screen golf course to capture the Y-Z plane in FIG. 1.

At this time, the Y-axis in FIG. 1 is the direction from the swing plate 150 and the tee box 151 toward the screen 155, the Z-axis is the height direction of the screen golf course, and the X-axis is the direction perpendicular to the Y-axis and Z-axis.

As illustrated in FIG. 2, the sensing unit 152 can transmit first image information about changes in position of the golf ball on the X-Y plane and second image information about changes in position of the golf ball on the Y-Z plane to the processing unit 130. The processing unit 130 can derive the trajectory of the golf ball by processing the first image information and the second image information and determining the change in coordinates of the golf ball in the X-Y-Z space.

Such a method of deriving the trajectory of the golf ball is just one example and the present invention is not limited thereto, and the trajectory of the golf ball can be derived by various methods. For example, the sensing unit 152 can derive the trajectory of the golf ball by sensing the location where the golf ball hits the screen 155 according to the user's swing.

The processing unit 130 processes a virtual golf game with the virtual player character based on the derived trajectory and the condition data of a past golf game, and makes the progress of the virtual golf game displayed on the screen 155.

The processing unit 130 can display a list of real professional golfers on the display unit or the screen 155 to allow the user to choose. When the user selects a preferred real professional golfer from the list, the processing unit 130 can proceed with a virtual golf game for the user and the virtual player character of the selected real professional golfer.

Furthermore, the processing unit 130 can display a list of past golf games, in which the real professional golfer participated, on the display unit or screen 155 to allow the user to choose. When the user selects one from the list of past golf games, the processing unit 130 can read the condition data of the selected past golf game from the virtual game storage unit 110 and implement a golf course displayed on the screen 155 according to the condition data.

As described above, the user can enjoy a game on a virtual golf course similar to the real golf course on which the real professional golfer played, together with the virtual player character of the real professional golfer. Consequently, the user can enjoy realistic screen golf.

Meanwhile, the condition data of the past golf game may include wind direction data and wind speed data of the past golf game. For example, the condition data of the past golf game may include wind direction data and wind speed data in the 2002 US Open at which Tiger Woods won.

The wind direction data and the wind speed data may be hole-by-hole wind direction/speed data, or wind direction/speed data by the date on which the past golf game was held, but is not limited thereto.

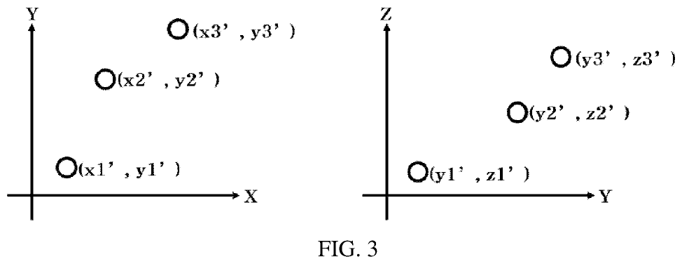

The processing unit 130 can change the trajectory of the golf ball, which was derived based on at least one of the wind direction data and the wind speed data. For example, if the trajectory of the golf ball according to the user's swing is the same as that illustrated in FIG. 2, the processing unit 130 can derive a changed trajectory as in FIG. 3 according to the wind direction data and wind speed data.

Accordingly, since the user may feel the actual environmental influence from the past golf game, the present invention makes the user feel realism as if the user is playing a golf game with a real professional golfer.

Meanwhile, the processing unit 130 can conduct a virtual golf game on a virtual course, which implements the teeing ground, the through the green, the green, and the hazards that constitute the holes of the past golf game.

As described above, the virtual game storage unit 110 of the present invention can store graphic information on locations and shapes of the teeing ground, the through the green, the green, and the hazards and landscape of each course for each of the 18 holes in the past golf games that famous professional golfers played, along with course landscape graphic information and so forth.

So, the processing unit 130 can implement a virtual course, and a virtual golf game can be conducted between the user and a virtual player character on the virtual course.

The user can experience a sense of reality as if the user are participating in the past golf game while playing the virtual golf game.

On the other hand, the processing unit 130 can apply a handicap to the virtual player character in the virtual golf game, to enable the user to compete with the virtual player character of the real professional golfer.

Because there is a significant skill gap between the real professional golfer and the user, a handicap to allow the user to enjoy the virtual game with the virtual player character of the real professional golfer may be applied to offset the skill gap.

In this case, the virtual game storage unit 110 can store accumulated results of many virtual golf games that the user participated in, and the processing unit 130 can evaluate the user's skill by analyzing the accumulated results, and adjust the size of the handicap based on the evaluation results.

Alternatively, the processing unit 130 can set the probability of victory for the virtual player character and process the virtual golf game. For example, even if the real professional golfer had a birdie or par save in a particular hole in the past golf game, the processing unit 130 can enhance the user's game fun by treating the virtual player character to have a bogey or double bogey in the particular hole in the virtual golf game, according to the victory probability.

Furthermore, the virtual game storage unit 110 can store relay voice data. The relay voice data may include voice data of broadcasters for pre-set events of a virtual golf game. The voice data of the broadcasters can be created by synthesizing the voice information of the broadcasters who relayed the past golf games.

The processing unit 130 can process the voice data of broadcasters when an event occurs during the processing of a virtual golf game. As a result, the voice data of the broadcasters can be outputted from the speaker 153 illustrated in FIG. 1.

The voice data of the broadcasters may include at least one of voice data of a caster and voice data of a commentator.

The event may occur during the progression of the virtual golf game. For example, the event may be related to a description of the features of each hole constituting the course, tee-up and tee-off, birdie, par save, and occurrence of bogey, the golf ball falling into a hazard or going out of bounds of the course, victory, defeat, and winning, but is not limited thereto.

Voice synthesis can artificially create voices by splitting the recorded voices of the broadcasters from broadcasts about the past golf games into certain voice units, attaching codes and inputting them into a synthesizer, and then recombining only the necessary voice units according to the instructions of the processing unit 130.

Additionally, it is also possible to perform voice synthesis based on recording the back part of the previous voice and the front part of the following voice centered on the boundary of the segmental sound of the voice. The present invention is not limited to such voice synthesis methods and can be achieved by various voice synthesis methods.

Such a voice synthesis method is just an example, and the present invention is not limited thereto.

Meanwhile, the processing unit 130 can set revenue for the actual professional golfer in proportion to the frequency of selection of the virtual player character by the user as an opponent in the virtual golf game.

In other words, there is a higher probability that the user may select virtual player characters of popular actual professional golfers, and the revenue is shared to the actual professional golfers of the virtual player characters with the higher frequency of selection, so the present invention motivates the actual professional golfers to actively participate in the virtual golf experience service provided by the present invention.

Meanwhile, the processing unit 130 can register the user's identification information and the number of strokes for each round in the virtual Hall of Fame when the user defeats the virtual player character of the actual professional golfer in the virtual golf game.

The user's identification information may include at least one among the user's name, user ID, user's facial image, and the user's swing images, but the present invention is not limited thereto.

Since the virtual golf game provided by the present invention is based on the past golf games that the actual professional golfers participated in, it may be difficult for the user to win against the virtual player characters. Therefore, the fact that the user has defeated the virtual player character indicates the user's excellent golf skills.

the present invention can induce other users to actively participate in the service of the present invention by registering users who have won the virtual golf game against virtual player characters in the virtual Hall of Fame.

Meanwhile, the processing unit 130 can read advertisement data featuring actual professional golfers who participate in the virtual golf game from the virtual game storage unit 110 and display the advertisement data on a screen unit 155.

The actual professional golfers are preferred advertisement models for advertisers as they can yield various marketing effects, resulting in a variety of advertisements featuring actual professional golfers being exposed to the public.

When the virtual golf game takes place between the user and the virtual player character, the processing unit 130 can enhance the marketing effect by displaying advertisement featuring the actual professional golfer, which serves as the basis for the virtual player character, through the screen unit 155.

Such advertisement data can be stored in the virtual game storage unit 110, and the processing unit 130 can request settlement of promotional costs to advertisers based on the exposure time of the advertisement data.

As described above, while the present invention has been particularly shown and described with reference to exemplary embodiments thereof, it will be understood by those of ordinary skill in the art that that the present invention can be embodied in other specific forms in addition to the above-described embodiments without departing from the spirit or scope of the present invention. Therefore, the above-described embodiments should be considered only as examples in all aspects and not for purposes of limitation, and the present invention is not limited to the above embodiments and may vary within the scope of the appended claims and their equivalents.

1. A screen golf device comprising a virtual game storage unit and a processing unit, wherein the virtual game storage unit stores data for virtual player characters of actual professional golfers and condition data of past golf games played by at least one of the actual professional golfers, and
wherein when a user strikes a golf ball on a tee box with a golf club on a swing plate, the processing unit:
derives the trajectory of the golf ball based on golf ball sensing information obtained from a sensing unit sensing the movement of the golf ball;
processes the virtual golf game between the virtual player character and the user based on the derived trajectory and the condition data of the past golf game; and
displays the progress of the virtual golf game on a screen unit.

2. The screen golf device according to claim 1, wherein the virtual game storage unit stores relay voice data,
wherein the relay voice data includes voice data of broadcasters for predetermined events of the virtual golf game,
wherein the voice data of the broadcasters is generated by synthesizing voice information of broadcasters who relayed the past golf game, and
wherein the processing unit processes the voice data of the broadcasters when the events occur during the processing of the virtual golf game.

3. The screen golf device according to claim 1, wherein the condition data of the past golf game includes wind direction data and wind speed data of the past golf game, and
wherein the processing unit changes the derived trajectory based on at least one of the wind direction data and the wind speed data.

4. The screen golf device according to claim 1, wherein the processing unit processes the virtual golf game in the virtual course implementing a teeing ground, a through the green, a green, and hazards that constitute the holes of the past golf game.

5. The screen golf device according to claim 1, wherein the processing unit applies a handicap to the virtual player character in the virtual golf game, to enable the user to compete with the virtual player character of the real professional golfer.

6. The screen golf device according to claim 1, wherein the processing unit sets revenue for the actual professional golfer in proportion to the frequency of selection of the virtual player character by the user as an opponent in the virtual golf game.

7. The screen golf device according to claim 1, wherein the processing unit registers the user's identification information and the number of strokes for each round in the virtual Hall of Fame when the user defeats the virtual player character of the actual professional golfer in the virtual golf game.

8. The screen golf device according to claim 1, wherein the processing unit reads advertisement data featuring actual professional golfers who participate in the virtual golf game from the virtual game storage unit and displays the advertisement data on the screen unit.

9. The screen golf device according to claim 2, wherein the processing unit processes the virtual golf game in the virtual course implementing a teeing ground, a through the green, a green, and hazards that constitute the holes of the past golf game.

10. The screen golf device according to claim 3, wherein the processing unit processes the virtual golf game in the virtual course implementing a teeing ground, a through the green, a green, and hazards that constitute the holes of the past golf game.
